(19) United States
(12) Patent Application Publication
(10) Pub. No.: US 2005/0054952 A1
(43) Pub. Date: Mar. 10, 2005

(54) Elongated medical device for intracorporal use

(75) Inventors: Alan D. Eskuri, Hanover, MN (US); Dave B. Johnson, Hopkins, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/656,945

(22) Filed: Sep. 5, 2003

(51) Int. Cl. A61M 25/09; A61B 5/00; B21F 45/00

(52) U.S. Cl. 600/585; 29/428; 140/71 R

(57) ABSTRACT

Alternative designs, materials and manufacturing methods for medical devices. Some embodiments relate to a medical device including two or more components or structures that are connected through heat crimping. In some embodiments, the heat crimping involves the use of a heat source to heat the material of one of the structures being connected to a point where it can flow or deform onto a surface of the other of the structures, and can thereafter be allowed to cool and form a mechanical bond between the two structures. In some embodiments, LASER energy is used as the heat source. Several alternative guidewire tip constructions and/or designs including methods and techniques of construction are also disclosed.

### FIELD OF TECHNOLOGY

The invention generally pertains to intracorporal medical devices, such as guidewires, catheters, or the like.

### BACKGROUND

A wide variety of medical devices have been developed for intracorporal use. Elongated medical devices are commonly used to facilitate navigation through and/or treatment within the anatomy of a patient. Because the anatomy of a patient may be very tortuous, it is desirable to combine a number of performance features in such devices. For example, it is sometimes desirable that the device have a relatively high level of pushability and torqueability, particularly near its proximal end. It is also sometimes desirable that a device be relatively flexible, particularly near its distal end. A number of different elongated medical device structures and assemblies, and methods of creating such structures and assemblies, are known, each having certain advantages and disadvantages. However, there is an ongoing need to provide alternative elongated medical device structures and assemblies, and methods of creating such structures and assemblies.

### SUMMARY OF SOME EMBODIMENTS

The invention provides several alternative designs, materials and methods of manufacturing alternative elongated medical device structures and assemblies. Some embodiments relate to a medical device including two or more components or structures that are connected together through heat crimping. For example, some embodiments relate to heat crimping of a first structure to a surface of a second structure.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic partial cross sectional view of a guidewire in accordance with one example embodiment;

FIG. 2 is a schematic close up partial cross sectional view of a portion of the guidewire of FIG. 1, showing a coil disposed about a core prior to attachment of the coil to the core;

FIG. 3 is a schematic partial cross sectional view of the portion of the guidewire as in FIG. 2, showing an energy source heating a portion of the coil;

FIG. 4 is a schematic partial cross sectional view of the portion of the guidewire as in FIG. 3, showing a portion of the coil attached to the outer surface of the core;

FIG. 5 is a schematic partial perspective view of an example embodiment of a guidewire including a coil attached to a core wire through a plurality of discrete connection areas that extend along a portion of the longitudinal axis of the core;

FIG. 6 is a schematic partial perspective view of an example embodiment of a guidewire including a coil attached to a core wire through a plurality of discrete connection areas that extend about the circumference of a portion of the coil;

FIG. 7 is a schematic partial cross sectional view of a portion of another embodiment of a guidewire similar to that shown in FIG. 1, but including a tubular member disposed about the core prior to attachment of the tubular member to the core;

FIG. 8 is a schematic partial cross sectional view of the portion of the guidewire as in FIG. 7, showing an energy source heating a portion of the tubular member;

FIG. 9 is a schematic partial cross sectional view of the portion of the guidewire as in FIG. 8, showing a portion of the tubular member attached to the outer surface of the core;

FIG. 10 is a schematic partial cross sectional view of another example embodiment of a guidewire construction similar to that shown in FIG. 1, and including a ribbon disposed on the distal end of the core;

FIG. 11 is schematic partial cross sectional view of another example embodiment of a guidewire construction similar to that shown in FIG. 7, and including a ribbon disposed on the distal end of the core; and

FIG. 12 is a schematic partial cross sectional view of another example embodiment of a guidewire construction similar to that shown in FIG. 1, and including a second coil member disposed on the first coil member.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EXAMPLE EMBODIMENTS

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention. For example, although discussed with specific reference to guidewires in the particular embodiments described herein, the invention may be applicable to a variety of medical devices that include two or more structures or assemblies connected together, and that are adapted to be advanced into the anatomy of a patient through an opening or lumen. For example, certain aspects of the invention may be applicable to fixed wire devices, catheters, such as therapeutic or diagnostic catheters (e.g. balloon, guide, infusion, stent delivery, etc.), drive shafts for rotational devices such as atherectomy catheters and IVUS catheters, endoscopic devices, laproscopic devices, embolic protection devices, spinal or cranial navigational or therapeutic devices, or the like, or components of any of these devices.

Some embodiments include a medical device including two or more components or structures that are connected together through heat crimping. Heat crimping can involve connecting two or more structures by using a heat source to heat a portion of a first structures such that at least a part of the heated portion deforms and/or flows onto the surface of a second structure. The heated portion is then allowed to cool, and solidify in a position on the surface of the second structure to create a mechanical bond between the two structures. The mechanical bond can be an interlocking bond or fit, or a frictional bond or fit.

In at least some embodiments, the bond is achieved by the deformation and/or flow of heated material from only one of the structures being connected. In some such embodiments, only a portion of a first structures is heated to a deformable and/or flowable state, for example, to its melting point. Therefore, the materials of the two structures do not intermix in a fluid state and fuse to a permanent union upon cooling. Additionally, in at least some embodiments, the mechanical bond is achieved without the use of a separate material, such as a solder, braze, or adhesive. Some other aspects of some examples of heat crimping will become apparent from the discussion of example embodiments below.

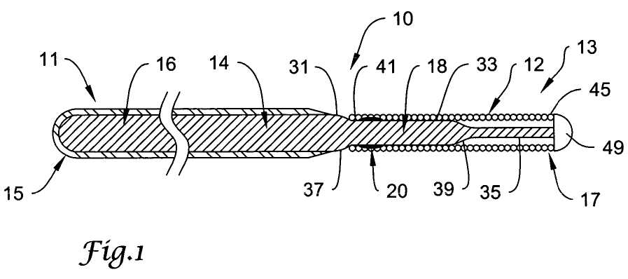

Refer now to FIG. 1, which is a partially cross-sectional view of an example medical device 10. In at least some embodiments, device 10 may be a guidewire, but as indicated above, other medical devices are contemplated. The guidewire 10 includes proximal guidewire region 11 and a distal guidewire region 13. The proximal region 11 includes proximal end 15, and the distal region 13 includes a distal end 17. The guidewire 10 includes a core member 14, in this embodiment, a core wire 14 including a proximal region 16 and a distal region 18. A structural member 12 is connected to the core member 14. In the embodiment shown, the structural member 12 is a coil member 12, such as a tubular coil member, connected to the core member 14 adjacent the distal region 13. The coil member 12 is connected to the core member 14 at one or more attachment areas 20, for example through heat crimping, as will be discussed in more detail below.

Those of skill in the art and others will recognize that the materials, structure, and dimensions of the core member 14 are dictated primary by the desired characteristics and function of the final guidewire, and that any of a broad range of materials, structures, and dimensions can be used. The following illustrates and describes some examples of such materials, structure, and dimensions of the core member 14, but it should be understood that others may be used.

The core member 14, including the proximal and distal regions 16/18, can be made of any suitable materials including metals, metal alloys, polymers, elastomers, such as high performance polymers, or the like, or combinations or mixtures thereof. Some examples of suitable metals and metal alloys include stainless steel, such as 304V, 304L, and 316L stainless steel; nickel-titanium alloy, such as linear elastic or superelastic (i.e., pseudoelastic) nitinol; nickel-chromium alloy, nickel-chromium-iron alloy, cobalt alloy, tungsten, tungsten alloy, tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), Elgiloy, hastelloy; monel 400; inconel 625; or the like; or other suitable material, or combinations or alloys thereof.

The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL). In some embodiments, nitinol alloys can include in the range of about 50 to about 60 weight percent nickel, with the remainder being essentially titanium. It should be understood, however, that in other embodiment, the range of weight percent nickel and titanium, and/or other trace elements may vary from these ranges. Within the family of commercially available nitinol alloys, are categories designated as “superelastic” (i.e. pseudoelastic) and “linear elastic” which, although similar in chemistry, exhibit distinct and useful mechanical properties to In some embodiments, a superelastic alloy, for example a superelastic nitinol can be used to achieve desired properties. Such alloys typically display a substantial “superelastic plateau” or “flag region” in its stress/strain curve. Such alloys can be desirable in some embodiments because a suitable superelastic alloy can provide a core member 14, or portion thereof, that exhibits some enhanced ability, relative to some other non-superelastic materials, of substantially recovering its shape without significant plastic deformation, upon the application and release of stress, for example, during insertion or navigation of the guidewire in the body.

In some other embodiments, a linear elastic alloy, for example a linear elastic nitinol can be used to achieve desired properties. For example, in some embodiments, certain linear elastic nitinol alloys can be generated by the application of cold work, directional stress, and heat treatment, such that the material fabricated does not display a substantial “superelastic plateau” or “flag region” in its stress/strain curve. Instead, in such embodiments, as recoverable strain increases, the stress continues to increase in a somewhat linear relationship until plastic deformation begins. In some embodiments, the linear elastic nickel-titanium alloy can be an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C., and in other embodiments, in the range of about −100° C. to about 100° C. The mechanical bending properties of such material are therefore generally inert to the effect of temperature over a broad range of temperature. In some particular embodiments, the mechanical properties of the alloy at ambient or room temperature are substantially the same as the mechanical properties at body temperature. In some embodiments, the use of the linear elastic nickel-titanium alloy allows the core member 14 to exhibit superior “pushability” around tortuous anatomy. One example of a suitable nickel-titanium alloy exhibiting at least some linear elastic properties is FHP-NT alloy commercially available from Furukawa Techno Material Co. of Kanagawa, Japan. Additionally, some examples of suitable nickel-titanium alloy exhibiting at least some linear elastic properties include those disclosed in U.S. Pat. Nos. 5,238,004 and 6,508,803, which are incorporated herein by reference.

In at least some embodiments, portions or all of core member 14, or other structures of the guidewire 10, may be doped with, made of, coated or plated with, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like.

In some embodiments, a degree of MRI compatibility is imparted into the core member 14, or other portions of the device 10. For example, to enhance compatibility with Magnetic Resonance Imaging (MRI) machines, it may be desirable to make core member 14, or other portions of the medical device 10, in a manner that would impart a degree of MRI compatibility. For example, core member 14, or portions thereof, may be made of a material that does not substantially distort the image and create substantial artifacts (artifacts are gaps in the image). Certain ferromagnetic materials, for example, may not be suitable because they may create artifacts in an MRI image. Core member 14, or portions thereof, may also be made from a material that the MRI machine can image. Some materials that exhibit these characteristics include, for example, tungsten, Elgiloy, MP35N, nitinol, and the like, and others.

The entire core member 14 can be made of the same material, or in some embodiments, can include portions or sections made of different materials. In some embodiments, the material used to construct core member 14 is chosen to impart varying flexibility and stiffness characteristics to different portions of core member 14. For example, proximal region 16 and distal region 18 may be formed of different materials, such as materials having different moduli of elasticity, resulting in a difference in flexibility. In some embodiments, the material used to construct proximal region 16 can be relatively stiff for pushability and torqueability, and the material used to construct distal region 18 can be relatively flexible by comparison for better lateral trackability and steerability. For example, proximal region 16 can be formed of straightened 304v stainless steel wire or ribbon, and distal region 18 can be formed of a straightened super elastic or linear elastic alloy, for example a nickel-titanium alloy wire or ribbon.

In embodiments where different portions of core member 14 are made of different material, the different portions can be connected using any suitable connecting techniques. For example, the different portions of the core wire can be connected using welding (including laser welding), soldering, brazing, adhesive bonding, heat or mechanical crimping, or the like, or combinations thereof. Additionally, some embodiments can include one or more mechanical connectors or connector assemblies to connect the different portions of the core wire that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. Nos. 09/972,276 entitled “GUIDEWIRE WITH STIFFNESS BLENDING CONNECTION” filed on Oct. 5, 2001, and Ser. No. 10/086,992 entitled “COMPOSITE GUIDEWIRE” filed on Feb. 28, 2002, both of which are incorporated herein by reference. Some additional examples of suitable interconnection techniques are disclosed in a U.S. patent application Ser. Nos. 10/375,766 entitled “COMPOSITE MEDICAL DEVICE” filed on Feb. 26, 2003, and Ser. No. 10/376,068 entitled “ELONGATED INTRACORPORAL MEDICAL DEVICE”, filed on Feb. 26, 2003, both of which are also incorporated herein by reference.

The length of core member 14 (and/or device 10), or the length of individual portions thereof, are typically dictated by the length and flexibility characteristics desired in the final medical device. For example, proximal region 16 may have a length in the range of about 20 to about 300 centimeters or more, distal region 18 may have a length in the range of about 3 to about 50 centimeters or more, and the guidewire 10 may have a total length in the range of about 25 to about 350 centimeters or more. It can be appreciated that alterations in the length of regions 16/18 can be made without departing from the spirit of the invention.

Core member 14 can have a solid cross-section, but in some embodiments, can have a hollow cross-section. In yet other embodiments, core member 14 can include a combination of areas having solid cross-sections and hollow cross sections. Moreover, core member 14, or portions thereof, can be made of rounded wire, flattened ribbon, or other such structures having various cross-sectional geometries. The cross-sectional geometries along the length of core member 14 can also be constant or can vary. For example, FIG. 1 depicts core member 14 as having a round cross-sectional shape. It can be appreciated that other cross-sectional shapes or combinations of shapes may be utilized without departing from the spirit of the invention. For example, the cross-sectional shape of core member 14 may be oval, rectangular, square, polygonal, and the like, or any suitable shape.

As shown in FIG. 1, distal region 18 may include one or more tapers or tapered regions. In some embodiments distal region 18 may be tapered and have an initial outside size or diameter that can be substantially the same as the outside diameter of proximal region 16, which then tapers to a reduced size or diameter. For example, in some embodiments, distal region 18 can have an initial outside diameter that is in the range of about 0.010 to about 0.040 inches, that tapers to a diameter in the range of about 0.001 to about 0.005 inches. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness.

In the embodiment shown in FIG. 1, the distal region 18 includes three constant diameter regions 31, 33, and 35, interconnected by two tapering regions 37 and 39. The constant diameter regions 31, 33, and 35 and tapering regions 37 and 39 are disposed such that the distal region 18 includes a geometry that decreases in cross sectional area toward the distal end thereof. In some embodiments, these constant diameter regions 31, 33, and 35 and tapering regions 37 and 39 are adapted and configured to obtain a transition in stiffness, and provide a desired flexibility characteristic. Also in some embodiments, portions of the distal region 18 can be flattened, for example, to provide for desired flexibility characteristics, or to provide an attachment area for other structure. For example, constant diameter region 35 could include a portion thereof that is flattened.

Although FIG. 1 depicts distal region 18 of core member 14 as being tapered, it can be appreciated that essentially any portion of core member 14 may be tapered and the taper can be in either the proximal or the distal direction. As shown in FIG. 1, the tapered region may include one or more portions where the outside diameter is narrowing, for example, the tapering regions 37 and 39, and portions where the outside diameter remains essentially constant, for example, constant diameter regions 31, 33, and 35. The number, arrangement, size, and length of the narrowing and constant diameter portions can be varied to achieve the desired characteristics, such as flexibility and torque transmission characteristics. The narrowing and constant diameter portions as shown in FIG. 1 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, core member 14 can be centerless ground using a Royal Master HI-AC centerless grinder. Some examples of suitable grinding methods are disclosed in U.S. patent application Ser. No. 10/346,698 entitled “IMPROVED STRAIGHTENING AND CENTERLESS GRINDING OF WIRE FOR USE WITH MEDICAL DEVICES” filed Jan. 17, 2003, which is herein incorporated by reference.

FIG. 1 also shows the structural member 12, which in this embodiment is a coil member 12 disposed about and connected to a portion of the distal region 18 of the core wire. It will be understood by those of skill in the art and others that a broad variety of materials, dimensions, and structures can be used to construct suitable embodiments of the structural member 12, depending upon the desired characteristics. The following examples are included by way of example only, and are not intended to be limiting.

The coil member 12 may be made of a variety of materials including metals, metal alloys, polymers, and the like, including those described above with regard to the core member 14. Some examples of some suitable materials include stainless steel, such as 304V, 304L, and 316L stainless steel; alloys including nickel-titanium alloy such as linear elastic or superelastic (i.e. pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material.

In some embodiments, the core member 14 can include multiple portions or layers wherein different portions or layers can include or be made of different materials. Additionally, the coil member 12 can be made of, coated or plated with, or otherwise include a radiopaque material and/or can include materials or structure to impart a degree of MRI compatibility, as discussed above in relation to the core member 14.

In at least some embodiments, the coil member 12, or a portion thereof, can be made of or include a material, such as a metallic material, that can be heated from a solid state to a state where it can flow, and thereafter can be allowed to cool and solidify. For example, in some embodiments, the metallic material of at least a portion of the member 12 may include a melting point temperature above which the material can be heated to flow in a liquid or semi-liquid form, and can thereafter be allowed to cool to a temperature below its melting point and solidify. In at least some embodiments, the member 12, or a portion thereof, can be made of or include a metallic material that can be heated such that it can flow at a temperature below a temperature at which the material used to construct at least a portion of the core member 14 will melt or flow, or otherwise be adversely affected. For example, in some embodiments, the coil member 12, or a portion thereof, can include a material that has a first predetermined melting or flowing point temperature, and the core member 14, or a portion thereof, can be made of or include a material that has a second predetermined melting or flowing point temperature that is above the first predetermined melting or flowing point temperature. For another example, in some embodiments, the core member 14, or a portion thereof, can be made of or include a material that has certain characteristics, such as flexibility, elasticity, torquability, or the like, that may be adversely affected when the material is exposed to certain predetermined temperatures, and the coil member 12 can include material that has a melting or flowing point temperature that is below this predetermined temperature. In at least some embodiments, the material used in at least a portion of the coil member 12 is not the same, or is dissimilar to the material used in at least a portion of the core member 14. Additionally, in some embodiments, the material used in at least a portion of the coil member 12 and the material used in at least a portion of the core member 14 are both metals or metal alloys.

The coil member 12 may be formed of round wire or flat ribbon ranging in dimensions to achieve the desired flexibility. It can also be appreciated that other cross-sectional shapes or combinations of shapes may be utilized without departing from the spirit of the invention. For example, the cross-sectional shape of wires or filaments used to make the coil may be oval, rectangular, square, triangle, polygonal, and the like, or any suitable shape. The size of the wires, ribbons, or filaments used to construct the coil member 12 can also vary, depending upon desired characteristics. In some embodiments, the coil member can include or be made of wires, ribbons, or filaments having a diameter in the range of about 0.001 to about 0.004 inches.

The coil member 12 can be wrapped in a helical fashion by conventional winding techniques. The pitch of adjacent turns of coil member 12 may be tightly wrapped so that each turn touches the succeeding turn or the pitch may be set such that coil member 12 is wrapped in an open fashion. The pitch can vary greatly, depending upon desired characteristics. In some embodiments, the coil member 12 can have a pitch in the range of up to about 0.05 inches, or in the range of up to about 0.02 inches, or in the range of about 0.001 to about 0.004 inches. The pitch can be constant throughout the length of the coil 12, or can vary, depending upon the desired characteristics, for example flexibility. These changes in coil pitch can be achieved during the initial winding of the wire, or can be achieved by manipulating the coil after winding or after attachment to the guidewire.

Additionally, in some embodiments, portions or all of the coil member 12 can include coil windings that are pre-tensioned or pre-loaded during wrapping, such that each adjacent coil winding can be biased against other adjacent coil windings to form a tight wrap. Such preloading could be imparted over portions of, or over the entire length of the coil member 12.

The size of the coil member 12 can also vary greatly, depending upon the desired characteristic, and the size of the other structures in the device 10, such as the core wire 14. The diameter of the coil member 12 can be sized to fit around and mate with a portion of the core member 14, and to give the desired characteristics, and can be constant and/or tapered. In some embodiments, the coil member 12 is tapered, for example, to mate with a tapered section of the core wire 14, or with other structure. The diameter of the coil member 12 can also include a taper beyond the distal end of the core member 14, as desired. In some embodiments, the coil member 12 can have an outer diameter that is in the range of about 0.01 to about 0.015 inches, and an inner diameter that is in the range of about 0.004 to about 0.013 inches.

The coil member 12 can be disposed about the core member 14 in any of a broad variety of configurations. In the particular embodiment shown, the coil member 12 can extend about a portion of the distal section 18 from a point adjacent the tapering region 37 distally to a point adjacent the distal most portion of the distal section 18. The coil member 12 is attached to the distal core wire section 16 at its proximal end 41 at one or more attachment areas, for example attachment area 20, using a suitable heat crimping attachment technique, or the like, as will be discussed below. The distal end 45 of the coil member 12 can be attached to the distal end of the core member 14 via a tip portion, for example, a rounded tip portion 49. The rounded tip portion 49 can be made of any suitable material, for example a solder tip, a polymer tip, a metal and/or metal alloy tip, or combinations thereof, or the like. Attachment to the tip portion 49 can be made using any suitable technique, including, for example, soldering, welding, heat crimping, adhesive, mechanical bonding or fitting, or combinations thereof, or the like. In some other embodiments, the distal end 45, or other portions of the coil member 12, may be attached to other structure, for example, ore or more spacer member, centering ring, additional coil, shaping or safety ribbon or wire, or may be free of attachment. Additionally, the coil member 12 can be attached to the core member 14 or other structure at one or more intermediate areas.

It should be understood, that these attachment areas are given by way of example only, and that the coil member 12 can be attached at different locations and by using more or fewer attachment areas, as desired, without parting from the spirit and scope of the invention. Additionally, in other embodiments, the coil member 12 can be disposed at other locations along the length of the guidewire 10, or could extend the entire length of the guidewire 10. In some embodiments, the coil member 12 can be in the range of about 1 to about 20 inches long.

As indicated above, attachment of the coil member 12 to the core member 14 at attachment area 20, or at other locations along the length of the core member 14, can be achieved using a heat crimping process. Heat crimping can involve the use of a heat source to heat a portion of a structure, in this case, a portion of the coil member 12, to a point where at least a part of the heated portion of the material of the coil member 12 deforms and/or flows onto the surface of the core member 14. The heated portion is then allowed to cool, and solidify in a position. At least the part of the material that is disposed on the surface of the core member 14 solidifies to create a mechanical bond between the two structures. The mechanical bond can be an interlocking bond or fit, or a frictional bond or fit.

Refer now to FIGS. 2-4 for a discussion of one example embodiment of attaching the coil member 12 to the core member 14. FIG. 2 is a close-up cross-sectional view of a portion of the guidewire 10 showing the coil member 12 disposed about the constant diameter portion 33 of the core member 14 prior to heat crimping of the coil member 12 to the core member 14. In the embodiment shown, the coil member 12 has an inner surface 40 and an outer surface 41, and the core member 14 has an outer surface 42. The coil member 12 is disposed about the core member 14 such that at least a portion of the inner surface 40 is in contact with at least a portion of the outer surface 42. However, in other embodiments, or at other areas along the length of the coil member 12, some spacing may occur between the surfaces 40 and 42.

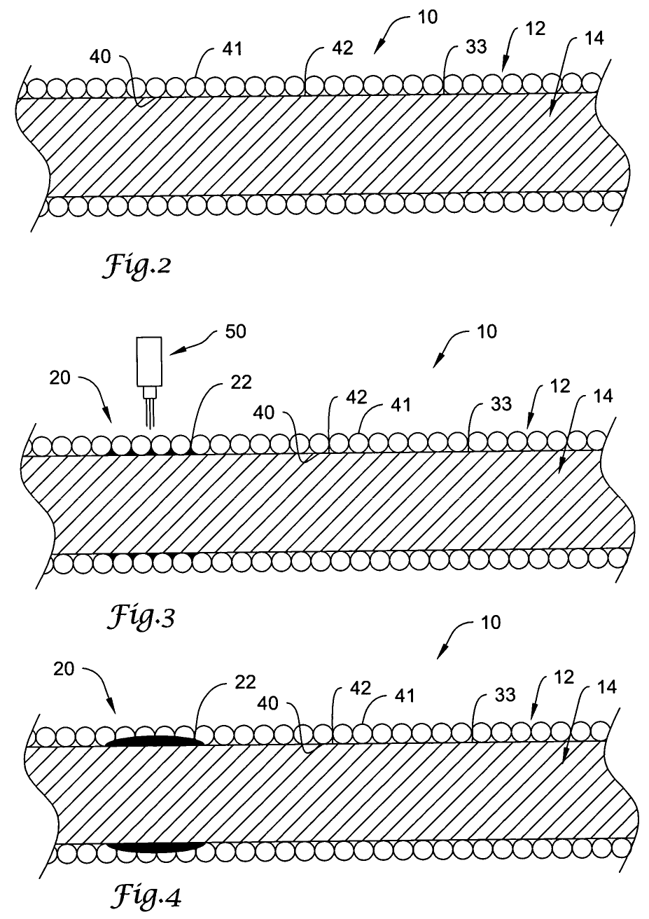

FIG. 3 is a close up view similar to that of FIG. 2, but showing a heating source 50 disposed adjacent a portion of the coil member 12. The heating source 50 is activated to provide energy to a portion of the coil member 12, and the energy results in the heating of a portion of the coil member 12. As the portion of the coil member 12 is heated to a predetermined temperature, at least a part of the heated portion of the coil member 12 begins to flow onto the surface 42 of the core member 14, and begins to form one or more connection areas 22 at attachment area 20. Either the core member 14 - coil member 12 assembly, and/or the heat source 50, or both, can be moved during the heating to heat different portions of the coil member 12 to achieve the desired size, shape, or other configuration to the connection area 22, or to create multiple connection areas 22. For example, the core member 14—coil member 12 assembly can be moved either laterally or rotationally in relation to the heating source 50 to provide heat to the desired portions of the coil member 12. Likewise, the heating source 50 can be moved laterally or circumferentially about the coil member 12 to provide heat to the desired portions of is the coil member 12.

In at least some embodiments, as the portion of the coil member 12 is heated to a predetermined temperature, the adjacent material of the core member 14 is not heated to a point where it can flow and intermix with the heated material of the coil member 12. This can be achieved, for example, by using a material for at least a portion of the core member 14 that has a melting point above that of the material used for the coil member 12. This may also be achieved by the accurate and/or careful application of the desired amount of heat to the desired areas, such as the portions of the coil, with out applying undue amounts of heat to the core wire 14. As such, heating can achieve the deformation and/or flow of material from only the coil member 12 onto the surface of the core member 14, and not the deformation and/or flow of material from the core member 14. Therefore, the materials of the two structures may not intermix in a fluid state.

After the desired portions of the coil member 12 have been heated to a point where a sufficient amount of material from the coil member 12 has flowed and/or deformed onto the surface 42 of the core member 14, the heat source can be removed and/or deactivated, as shown in FIG. 4. As shown in FIG. 4, at least a part of the heated portion of the coil member may remain intact or in contact within the structure of the coil, while a part flows onto the surface of the core member 14. The heated material from the coil member 12 can be allowed to cool. As it cools, a portion thereof is disposed on and solidifies in a position on the surface 42 of the core member 14 to create a connection area 22 that includes mechanical interface or bond between the coil member 12 and the core member 14. The mechanical bond or interface can be an interlocking bond or fit, or a frictional bond or fit. As such, the coil member 12 has been connected to the core member 14 at attachment area 20 via heat crimping.

Additionally, in at least some embodiments, the bond is achieved without the intermixing of flowable or molten materials from the core member 14 with the material of the coil member 12, and therefore the bond is achieved without the materials of the two structures fluidly intermixing and fusing to a permanent union upon cooling, for example, as may occur in the formation of a weld structure. In at least some embodiments, the material of the core member 14 never melts or flows, so such intermixing of materials cannot occur. Additionally, in at least some embodiments, the bond is achieved without the use of a separate material, such as a solder, braze, or adhesive. In some respects, a portion of the coil member 12 has been heated to flow onto and form a mechanical bond, or in essence, form a “crimp” on the outer surface of the core member 14.

Any of a number of heating sources can be used to create the energy used to heat the material of a portion of the coil member 12. However, in some embodiments, a relative degree of accuracy and small size in the heat source is used. For example, in some embodiments, a narrower, or more controlled heat source can be used, for example, a LASER energy source, to heat the desired portions of the coil member 12. In LASER crimping, a light beam is used to supply the necessary heat. LASER crimping can be beneficial, as the use of a LASER light heat source can provide a high degree of accuracy. The area affected by a LASER energy source can be adapted to be narrow to achieve the desired amount of accuracy. The use of LASER energy may be desirable to avoid undesirably heating larger areas surrounding the attachment area 20. For example, some heat sources may undesirably heat the entire area surrounding the attachment area. For example, if some of the components of the guidewire are heat sensitive materials, the heat may adversely affect the characteristics of the material. One example of such materials include some nickel titanium alloys, which if exposed to undue heat above a certain point, may undergo a phase change, or may anneal, which may effect the desired properties of the material. Additionally, less accurate heat sources may not allow for desirable control of the size and shape of the bonding area. Any of a variety of LASER sources can be used, depending upon the desired size and degree of accuracy. One example of a source of LASER energy includes a LASER diode, for example, a LASER diode used in LASER diode soldering. Another example of a source of LASER energy includes LASER welding equipment. LASER welding equipment which may be suitable in some applications is commercially available from Unitek Miyachi of Monrovia, Calif. and Rofin-Sinar Incorporated of Plymouth, Mich. It should be understood, however, that although such equipment may be used in welding and/or soldering applications, in the context of at least some embodiments of the invention, such equipment is used as a heating source to create the energy used to heat the material of a portion of the coil member 12, and is not necessarily used to create a weld or solder joint between the coil member 12 and the core member 14.

It is contemplated that other heating sources may be used, for example, sources that use plasma, light, RF, IR, electrical, friction, electron beam, radiant energy, or the like, may be used as a source of energy to create the necessary heating of a portion of the coil member 12. In some embodiments, such heat sources may be adapted to provide a desired degree of accuracy.

In some embodiments, one or more of the connection areas 22 created through heat crimping can extend around the entire perimeter, for example about the circumference, of a portion of the coil 12. In some other embodiments, however, one or more of the connection areas 22 can extend about only a part of the perimeter, for example, about only a portion of the circumference, of a portion of the coil 12.

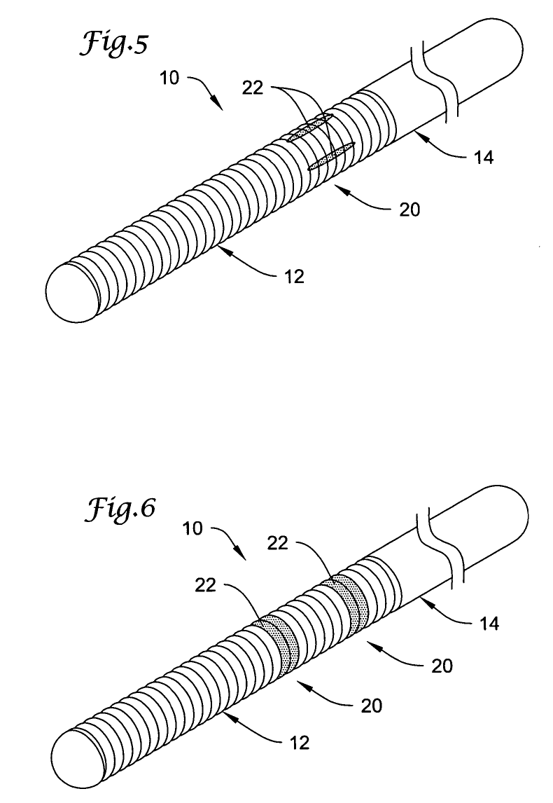

For example, refer to FIG. 6, which is a partial perspective view of another guidewire 10 similar to that shown in FIG. 1, including one or more connection areas 22 that do extend all the way around the circumference of a portion of the coil member 12.

For another example, refer to FIG. 5, which is a partial perspective view of a guidewire 10 similar to that shown in FIG. 1, including one or more connection areas 22 that extend longitudinally along a portion of the longitudinal axis of a portion of the coil member 12, but that do not extend all the way around the perimeter of a portion of the coil member 12. Multiple connection areas 22 are shown that are spaced from one another about the perimeter of the coil member 14, but other arrangement may be used. For example, the connection areas 22 may be longitudinally spaced from each other, or may be spaced from each other both longitudinally and circumferentially.

The number of connection areas 22, and the size and shape of each of the connection areas 22 can vary greatly, depending somewhat at least upon the desired characteristics of the connection and/or the desired characteristics of the guidewire 10. In some example embodiments, the coil 12 may include in the range of about 1 to about 20, or possibly more, such connection areas 22. In some embodiments, for example, where the connection areas 22 do not extend around the entire perimeter of the coil member, each individual connection area 22 may have a length (along the longitudinal axis) in the range of about 0.005 to about 0.025 inches, and/or a width in the range of about 0.005 to about 0.025 inches. Additionally, and/or alternatively, in embodiments where the connection areas 22 do extend around the entire perimeter of a portion of the coil member, each individual connection area 22 may have a width in the range of about 0.005 to about 0.025 inches. It should be understood that these dimensions are given by way of example only, and that they may vary from the ranges given in other embodiments. The length and width of the connection areas 22 on a particular construction may be the same, or may vary from one another, if more than one connection area is present.

As can be appreciated, the connection can be made using one or more discrete connection areas 22 as opposed to attachment of the entire length of the coil member 12 to the core member 14. For example, the discrete connection areas 22 may take up less than about 20%, or less than about 10%, less than about 5%, or less than about 2% of the entire area of the coil member 12 surface. In some embodiments, each individual connection area 22 may be disposed such that it encompasses or includes a limited number of coil windings from the coil member 12. For example, in some embodiment, the connection areas may encompass or include less than 25, less than 15, less than 10, or less than 5 coil windings. The use of certain heat sources, for example LASER heat sources, or the like, can be useful in making such discrete connection areas 22 because they tend to allow the accuracy needed to make such connections.

Additionally, the connection areas 22, for example those shown in FIGS. 5 and 6, may be disposed at certain locations and/or in certain density pattern that may achieve desirable flexibility, torquability, or other characteristics in the guidewire 10. For example, certain desired characteristics of the core member 14 and/or coil member 12 may be achieved by disposing the connection areas 22 at particular locations and/or in particular density patterns along the length of the guidewire 10.

Additionally, heat crimping techniques may be used to achieve desirable characteristics in coil member 12 itself by joining two or more coil windings within the coil member 12 together, either alone, or in combination with connection to the core wire 14. For example, the connection areas 22 disclosed above act to connect the coil member 12 to the core member 14, and in addition act to make a connection between adjacent coil windings within the coil member 12. In some embodiments, however, heat crimping techniques may be used to connect two or more coil windings together within the coil member 12 independently of connection of the coil windings to the core member 14. As such, such heat crimping can be used to achieve desired characteristics, such as flexibility and torque transmission characteristics, within the coil member 12 without connection to the core member. Some examples of joining coil windings together on a coiled member, and density patterns that can be used, to achieve desirable characteristics such as flexibility and/or torque transmission characteristics are disclosed in U.S. patent application Ser. No. entitled “MEDICAL DEVICE COIL” filed on even date herewith (Atty. Docket No. 1001.1675101); and U.S. patent application Ser. No. entitled “MEDICAL DEVICE COIL” filed on even date herewith (Atty. Docket No. 1001.1674101), both of which are incorporated herein by reference. In some embodiments, such coiled structures can be achieved using the heat crimping techniques disclosed herein.

In some embodiments, the structures being connected can be pre-treated and/or include structure that may aid in the formation and/or strength of the mechanical bond created through heat crimping. For example, the coil 12, or portions thereof, can be cleaned or treated to remove impurities or oxides to allow for a better flow of material. Additionally, the surface 42 of the core member 14, or portions thereof, may be mechanical, chemically, or otherwise treated or worked to create a rough or less smooth surface, or the like, which may provide for a better mechanical interlock or frictional fit with the material that flows from the coil member 12. Additionally, the outer surface of the core member 14, or portions thereof, may include one or more additional structure defined therein, such as a groove, notch, channel, indentation, furrow, cut, scratch, protrusion, flange, lip, outcropping, protuberance, or the like, which may provide for a better mechanical interlock or frictional fit with the material that flows from the coil member 12.

It should also be understood that the above described heat crimping techniques are merely illustrative, and that other suitable heat crimping techniques or structures can be used. Additionally, the heat crimping techniques described above can be used at other locations along the length of the guidewire, or can be used to attach other components of the guidewire to each other. For example, the member 12 connected using heat crimping may not be a coil, but may include other structures that can be incorporated into the construction of the device 10. For example, such an attachment method and/or technique can be used to attach coils, ribbons, braids, wires, centering rings, or the like, or other such structures to the proximal and/or distal regions of the core wire, or other structures of the guidewire 10. Additionally, such structures and methods can be used in the construction of other medical devices. For example, a coil member 12, or the like, could be heat crimped onto the distal portion of another medical device, such as a fixed wire device, a catheter, such as therapeutic or diagnostic catheter, a drive shaft for a rotational device, an endoscopic or laproscopic device, an embolic protection device, a spinal or cranial device, or the like.

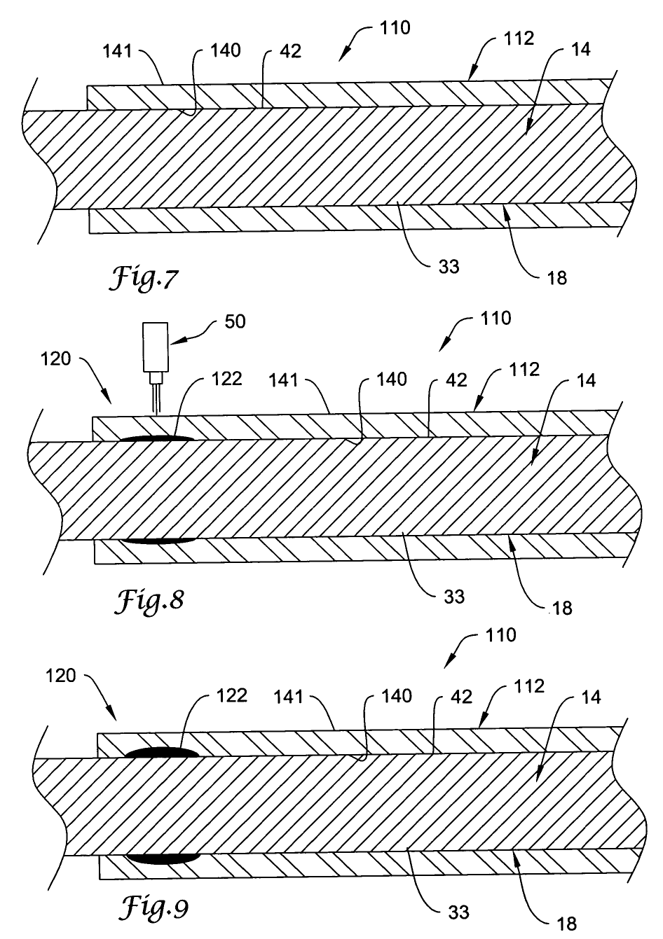

For example, FIGS. 7-9 show close up views of a guidewire 110 similar to that shown in FIG. 2-4, wherein like reference numbers indicate similar structure. In this embodiment, however, the structure or member to be connected to the core member 14 includes a tubular sleeve member 112 that is disposed about the core member 14. The sleeve member 112 can be any of a wide variety of structures, such as a hypotube, or other such structure that may or may not include additional structures, such as grooves, notches, protrusions, of the like defined therein. The sleeve member 112 can be disposed about a distal region 18 of the core member 14 in a similar manner as the coiled member 12 extends on the core member 14, as shown in FIG. 1. In other embodiments, the sleeve member 112 can extend further in a proximal direction, and in some cases can extend over the proximal guidewire section. In yet other embodiments, the sleeve member 112 can begin at a point distal of the tapered region.

Suitable material for use in the sleeve member 112 can include any material that would give the desired strength, flexibility or other desired characteristics. Some suitable materials include metals, metal alloys, polymers, and/or like material, for example, the material discussed above with regard to the core member 14 and the coil member 12.

Again, in some embodiments, the sleeve member 112, or a portion thereof, can be made of or include a material, such as a metallic material, that can be heated from a solid state to a state where it softens and can flow, and thereafter can be allowed to cool and solidify, as discussed in more detail above regarding the coil member 12. Additionally, in at least some embodiments, the member 112, or a portion thereof, can be made of or include a material that can melt and/or flow at a temperature below the temperature at which the material of the core member 14 will melt or flow, or otherwise be adversely affected.

The sleeve member 112 can be disposed around and attached to the core member 14 using a heat crimping method, similar to that discussed above. FIG. 7 is a the close-up cross-sectional view of a portion of the guidewire 10 showing the sleeve member 112 disposed about the constant diameter portion 33 of the core member 14 prior to heat crimping of the sleeve member 112 to the core member 14. In the embodiment shown, the sleeve member 112 has an inner surface 140 and an outer surface 141, and the core member 14 has an outer surface 42. The sleeve member 112 is disposed about the core member 14 such that at least a portion of the inner surface 140 is in contact with at least a portion of the outer surface 42. However, in other embodiments, or at other areas along the length of the sleeve member 112, some spacing may occur between the surfaces 140 and 42.

FIG. 8 is a close up view similar to that of FIG. 7, but showing a heating source 50 disposed adjacent a portion of the sleeve member 112. The heating source 50 is activated to provide energy to a portion of the sleeve member 112, and the energy results in the heating of a portion of the sleeve member 112. As the portion of the sleeve member 112 is heated to a predetermined temperature by the heating source 50, a part of the heated portion of the sleeve member 112 begins to melt and/or flow onto the surface 42 of the core member 14, and begins to form a connection area 122 at attachment area 120. Again, either the core member 14—sleeve member 112 assembly, and/or the heat source 50 can be moved during the heating to heat different portions of the sleeve member 112 to achieve the desired size, shape, or other configuration to the connection areas 22, and/or to create multiple connection areas 22, as discussed above with regard to the embodiments shown in FIGS. 2-4.

In at least some embodiments, as the portion of the sleeve member 112 is heated to a predetermined temperature, the adjacent material of the core member 14 is not heated to a point where it can flow and/or intermix in a fluid state with the heated material of the sleeve member 112. As such, the bond at the attachment area is achieved by the deformation and/or flow of material from only the sleeve member 112 onto the surface of the core member 14. Therefore, the materials of the two structures do not intermix in a fluid state.

After the desired portions of the sleeve member 112 have been heated to a point where a sufficient amount of material from the sleeve member 112 has flowed and/or deformed onto the surface 42 of the core member 14, the heat source can be removed and/or deactivated, as shown in FIG. 9. The heated material from the sleeve member 112 can be allowed to cool. As the heated material from the sleeve member 112 cools, a portion thereof is disposed on the surface of the core member 14, and solidifies in a position on the surface 42 of the core member 14 to create a mechanical interface or bond between the sleeve member 112 and the core member 14. The mechanical bond or interface can be an interlocking bond or fit, or a frictional bond or fit. As such, the sleeve member 112 has been connected to the core member 14 at attachment area 120 via heat crimping.

It should be understood that other embodiments of medical devices, such as guidewires, in accordance with the invention may include alternative constructions or additional structures, such as alternative tip constructions, additional wires or ribbons, such as safety and/or shaping ribbons (coiled or uncoiled), centering or attachment sleeves and/or structures, radiopaque markers, such as coils or bands, and the like, or other such structures. Such additional structures and components, in some embodiments, may be connected to the medical device using heat crimping techniques as disclosed herein, or using other connection techniques. Some examples of additional components and constructions for use in medical devices, such as guidewires, and the like, are disclosed in U.S. patent application Ser. Nos. 09/972,276 entitled “GUIDEWIRE WITH STIFFNESS BLENDING CONNECTION” filed on Oct. 5, 2001; Ser. No. 10/086,992 entitled “COMPOSITE GUIDEWIRE” filed on Feb. 28, 2003; and Ser. No. 10/376,068 entitled “ELONGATED INTRACORPORAL MEDICAL DEVICE” filed on Feb. 26, 2003, all of which are incorporated herein by reference.

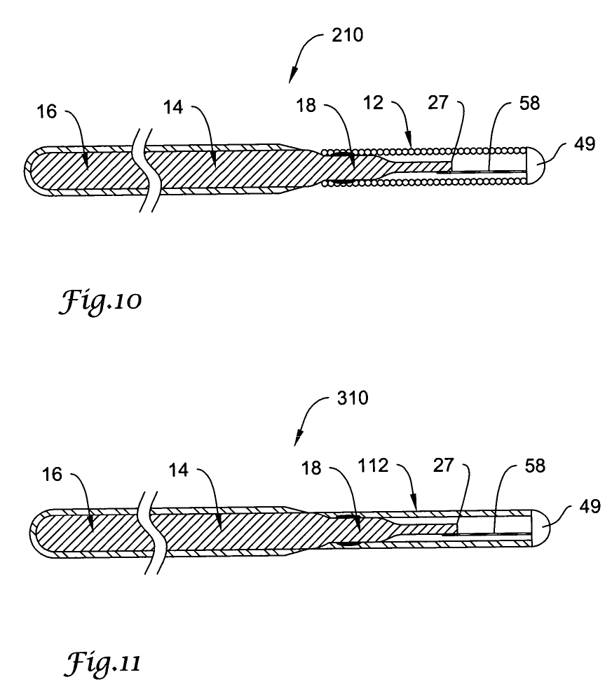

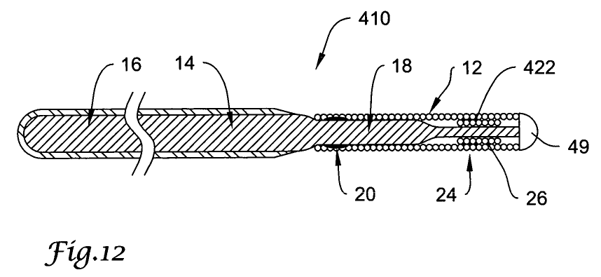

For example, FIGS. 10 and 11 show embodiments of guidewires 210 and 310, respectively, that are similar to the construction shown in FIG. 1, wherein like reference numbers indicate similar structure. However, in these embodiments, the guidewires 210 and 310 include an alternative tip construction, including a wire or ribbon 58 that is attached adjacent the distal end 27 of the distal section 18 of the core member 14, and extends distally of the distal end 27. FIG. 10 shows a tip construction including a ribbon 58 in an embodiment of a guidewire 210 including a coil member 12 construction similar to that described above in relation to FIGS. 1-6, while FIG. 11 shows a tip construction including a ribbon 58 in an embodiment of a guidewire 310 including a tubular sleeve member 112 construction similar to that described above in relation to FIGS. 7-9. In the embodiments shown in FIGS. 11 and 12, however, the coil 12 and sleeve 112, respectively, extend distally beyond the distal end 27 of the core member 14.

In some embodiments, the wire or ribbon 58 can be a fabricated or formed wire structure, for example a coiled wire. In the embodiments shown however, the ribbon 58 is a generally straight ribbon that overlaps with and is attached to the distal end 27 of the core member 14.

The ribbon 58 can be made of any suitable material and sized appropriately to give the desired characteristics, such as strength and flexibility characteristics. Some examples of suitable materials include metals, metal alloys, polymers, and the like, and may include radiopaque materials or include materials or structure to impart a degree of MRI compatibility, as discussed above in relation to the core member 14 and coil member 12. The ribbon 58 can be attached to the distal section 18 using any suitable attachment technique. Some examples of attachment techniques include heat crimping, soldering, brazing, welding, adhesive bonding, mechanical crimping, or the like. In some embodiments, the ribbon or wire 58 can function as a shaping structure or a safety structure. The distal end of the ribbon 58 can be free of attachment, or can be attached to another structure, for example the tip portion 49, as shown.

Refer now to FIG. 12, which shows another example embodiment of a guidewire 410 very similar to that shown in FIG. 1, wherein like reference numerals indicate similar structure as discussed above. The core member 14 and the coil member 12 can include the same general construction, structure, materials, and methods of construction and attachment as discussed above with regard to like components in the embodiments of FIG. 1. However, in this embodiment, the guidewire 410 also includes an inner coil member 26 connected to the coil member 12 to form a dual coil tip construction.

In the embodiment shown, the inner coil 26 is disposed about the distal section 18 of the core member 14 about a portion of the constant diameter section 35, and is disposed within the lumen of the coil member 12, however, in other embodiments, other configurations may be used. The inner coil 26 can be made of the same materials, and have the same general construction and pitch spacing as discussed above with regard to the coil member 12. The inner coil 26, however, would include an outer diameter that allows it to fit within the lumen of the coil member 12, and in some embodiments, has an outer diameter that allows it be disposed in contact with, and in some cases, have a relatively snug or tight fit with the inner diameter of the coil member 12. In some embodiments, the inner coil 26 can be made of a radiopaque material, for example, a platinum/tungsten wire, while the coil member 12 is made of a less radiopaque material, for example, MP35-N, or vice versa. It will be understood by those of skill in the art and others that a broad variety of materials, dimensions, and structures can be used to construct suitable embodiments, depending upon the desired characteristics. The following examples are included by way of example only, and are not intended to be limiting. The inner coil 26 can be in the range of about 0.1 to about 3 inches long, and is made of rounded wire having a diameter of about 0.001 to about 0.005 inches. The coil 26 can have an outer diameter that is generally constant, and is in the range of about 0.002 to about 0.015 inches. The inner diameter of the coil can also be generally constant, and is in the range of about 0.001 to about 0.008 inches. The pitch of the coil 26 can be in the range of about 0.0005 to about 0.04 inches.

The coil 26 is attached to the outer coil member 12 at attachment area 24, for example, using a heat crimping technique. The distal end 97 of the coil 26 can be free of attachment. However, in other embodiments, distal end 97 of the coil 26 can be attached to the coil member 12, or can be attached to other structure, for example, to the tip portion 49, to the core member 14, to a centering or attachment ring, or other such structure. In some particular embodiments, the inner coil 26 is attached only to the outer coil member 12 at one or more attachment areas, and is essentially free of any other connection to a core member 14, or in some cases, is free of connection to any other structure in the guidewire 410 other than the outer coil member 12. Additionally, the inner coil 26 can be attached to the outer coil member 12 along the entire length of the inner coil 26, or only along a portion of the length thereof. For example, in the embodiment shown, the inner coil 26 is attached only at the proximally disposed attachment area 24. In other embodiments, the coil 26 may be attached using other arrangements, for example, a distally disposed attachment area, or a combination of proximally and distally disposed attachment areas. Attachment of the inner coil 26 to the outer coil member 12 can be achieved using any suitable heat crimping attachment technique, as discussed above, for example using LASER energy, to heat the outer coil member 12 such that material flows there from, and acts to attach it to the inner coil member 26.

In some embodiments, the attachment of the inner coil 26 to the outer coil member 12 can be achieved by forming one or more connection areas 422 at attachment area 24 that extend around the entire perimeter of the coils 12 and 26. In some other embodiments, however, one or more spaced connection areas 422 that do not extend all the way around the perimeter of the coils 12 and 26 can be made. The connection areas 422 may be longitudinally and/or circumferentially spaced from each other, or both.

As discussed above, the number, size, shape, location, and/or density pattern of the connection areas 22 can vary greatly, depending somewhat at least upon the desired characteristics of the connection and/or the desired characteristics of the guidewire 10. The number, size, shape, location, and/or density pattern of the connection areas 422 can be similar to the connection areas 22 discussed above, and may be adapted and/or configured to achieve at least some of the came characteristics.

Again, it can be appreciated that the connection areas 422 can be discrete connection areas 422 as opposed to attachment of the entire length of the coil member 12 to the coil member 26. For example, the discrete connection areas 22 may take up less than about 20%, or less than about 10%, less than about 5%, or less than about 2% of the entire area of the coil member 12 surface. In some embodiments, each individual connection area 22 may be disposed such that it encompasses or includes a limited number of coil windings from the coil member 12 and/or the coil member 26. For example, in some embodiment, the connection areas may encompass or include less than 25, less than 15, less than 10, or less than 5 coil windings from wither of the coil members.

As discussed above, in some particular embodiments, the inner coil 26 is attached only to the outer coil member 12 at one or more attachment areas, and is essentially free of any other connection to a core wire 14, or in some cases, is free of connection to any other structure in the guidewire 410. Some such embodiments can provide the benefit of one or more additional coils, for example coil 26, disposed within the guidewire structure without the need to attach such coils to a shaft or core wire. For example, in some cases, it may be undesirable to attach additional structures to a core or shaft portion of a guidewire due to the possible changes in the flexibility or other characteristics at an attachment area. Thus, it may be desirable to avoid such attachment areas, and attach any additional coils to a coil that is attached to the core wire or shaft, such as the outer coil member 12.

Such an arrangement of an inner coil being attached only to an outer coil could be used in a broad variety of medical devices. Some example of coil constructions that can be used in a broad variety of medical devices are disclosed in U.S. patent application Ser. No. 10/376,068 entitled “ELONGATED INTRACORPORAL MEDICAL DEVICE” filed on Feb. 26, 2003, which is incorporated herein by reference. Such coil constructions disclosed therein can also be achieved by using the heat crimping techniques disclosed herein.

Additionally, in some embodiments, a coating, for example a lubricious (e.g., hydrophilic) or other type of coating may be applied over portions or all of the medical devices, and/or structures discussed above. For example, such a coating may be applied over portions or all of a device, for example guidewires 10, 110, 210, 310, and 410, including, for example, core wire sections 16/18, the coil or sleeve members 12 or 112, the distal tip 69, or other portions of the guidewires. In the embodiments shown in FIGS. 1, 10, 11, and 12, a coating 61 is disposed over a proximal portion of the guidewire. Hydrophobic coatings such as fluoropolymers, silicones, and the like provide a dry lubricity which improves guide wire handling and device exchanges. Lubricious coatings improve steerability and improve lesion crossing capability. Suitable lubricious polymers are well known in the art and may include hydrophilic polymers such as, polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. In some embodiments, the more distal portion of the guidewire is coated with a hydrophilic polymer as discussed above, and the more proximal portions is coated with a fluoropolymer, such as polytetrafluroethylene (PTFE).

It should be understood that this disclosure is, in many respects, only illustrative. Changes may be made in details, particularly in matters of shape, size, and arrangement of steps without exceeding the scope of the invention. For example, heat crimping techniques as disclosed herein can be used in a broad variety of medical devices, and can be used to connect alternative structure. Additionally, alternative tip constructions including a flexible coil tip, a polymer jacket tip, a tip including a coiled safety/shaping wire, or combination thereof, and other such structure may be placed on the guidewire. The invention's scope is, of course, defined in the language in which the appended claims are expressed.

1. A method of making a medical device, the method comprising:
providing an elongated shaft defining a surface;
providing a structural member comprising a metallic material having a predetermined melting point temperature above which the material can flow;
disposing the structural member adjacent the elongated shaft such that at least a portion of the structural member is adjacent the surface;
heating a discrete portion of the structural member to a temperature at or above the predetermined melting point temperature;
allowing the heated portion of the structural member to flow onto the surface of the elongated shaft; and
allowing the heated portion of the structural member to cool on the surface of the elongated shaft such that a discrete connection area is created forming a mechanical bond between the structural member and the elongated shaft.

2. The method of claim 1, wherein the structural member comprises a tubular member defining a lumen and having an inner surface, and the disposing includes disposing the structural member about the elongated shaft such that at least a portion of the elongated shaft is disposed within the lumen of the structural member.

3. The method of claim 2, wherein the structural member comprises a helical coil.

4. The method of claim 2, wherein the structural member comprises a tubular member.

5. The method of claim 2, wherein the tubular member includes a perimeter, and the discrete connection area extends about the perimeter of a portion of the tubular member.

6. The method of claim 2, wherein the tubular member includes a perimeter, and the discrete connection extends about only a portion of the perimeter of a portion of the tubular member.

7. The method of claim 1, wherein the elongated shaft comprises a material having a second predetermined melting point temperature above which the material can flow, and the second predetermined melting point temperature is greater than the first predetermined melting point temperature.

8. The method of claim 1, wherein the elongated shaft comprises a material having characteristics that are adversely affected when exposed to a second predetermined temperature, and the second predetermined temperature is greater than the first predetermined melting point temperature.

9. The method of claim 1, wherein the elongated shaft comprises a metallic material.

10. The method of claim 1, wherein the elongated shaft comprises stainless steel or a nickel-titanium alloy, and wherein the structural member comprises a nickel-titanium alloy, tungsten, platinum, MP35-N, elgiloy, hastelloy, or combinations or alloys thereof.

11. The method of claim 1, wherein the heating of the discrete portion of the structural member comprises heating with LASER energy.

12. The method of claim 1, wherein the elongated shaft comprises a material having a melting point, and the heating step includes avoiding heating of the material of the elongated shaft to the melting point thereof.

13. The method of claim 1, wherein the elongated shaft comprises a material, and wherein the discrete connection area is created to form the mechanical bond between the structural member and the elongated shaft is achieved without the intermixing in a fluid state of material from the elongated shaft and material from the structural member.

14. The method of claim 1, wherein the discrete connection area is created to form the mechanical bond between the structural member without the use of an additional bonding material.

15. The method of claim 1, wherein a plurality of discrete connection areas are created to form the mechanical bond between the structural member and the elongated shaft.

16. A method of making a medical device, the method comprising:
providing an elongated shaft comprising a material and defining a surface;
providing a structural member comprising a metallic material having a predetermined melting point temperature above which the material can flow;
disposing the structural member adjacent the elongated shaft such that at least a portion of the structural member is adjacent the surface;
heating a portion of the structural member to a temperature at or above the predetermined melting point temperature;
allowing at least a part of the heated portion of the structural member to flow onto the surface of the elongated shaft; and
allowing the heated portion of the structural member to cool on the surface of the elongated shaft such that a mechanical bond is formed between the structural member and the elongated shaft, wherein the mechanical bond is achieved without the intermixing in a fluid state of material from the elongated shaft and material from the structural member.

17. The method of claim 16, wherein the mechanical bond is achieved without the use of an additional bonding material.

18. The method of claim 16, wherein the elongated shaft comprises a material having a second predetermined melting point temperature, and wherein heating includes heating a discrete portion of the structural member to a temperature at or above the predetermined melting point temperature while avoiding heating the elongated shaft to the second predetermined melting point temperature; and the heated portion of the structural member is allowed to cool on the surface of the elongated shaft such that a discrete connection area is created forming a mechanical bond between the structural member and the elongated shaft.

19. A method of making a medical device, the method comprising:
providing an elongated shaft defining a surface;
providing a structural member comprising a material having a predetermined melting point temperature above which the material flows;
disposing the structural member on the elongated shaft such that at least a portion of the structural member is adjacent the surface;
using LASER energy to heat the portion of the structural member to a temperature at or above the predetermined melting point temperature;
allowing the heated portion of the structural member to flow onto the surface of the elongated shaft; and
allowing the heated portion of the structural member to cool on the surface of the elongated shaft such that a mechanical bond is formed between the structural member and the elongated shaft.

20. The method of claim 19, wherein the mechanical bond is achieved without the intermixing in a fluid state of material from the elongated shaft and material from the structural member.

21. The method of claim 19, wherein the structural member comprises a metallic material.

22. A method of making a guidewire, the method comprising:
providing an elongated core wire defining an outer surface;
providing a tubular member defining a lumen and having an inner surface, the tubular member comprising a metallic material having a predetermined melting point temperature above which the material can flow;
disposing a portion of the elongated core wire within the lumen of the tubular member such that at least a portion of the inner surface of the tubular member is adjacent the outer surface of the core wire;
heating a portion of the tubular member to a temperature at or above the predetermined melting point temperature of the metallic material;
allowing a part of the heated portion of the tubular member to flow onto the outer surface of the core wire; and
allowing the heated portion of the tubular member to cool such that the part disposed on the outer surface of the core wire forms a mechanical bond between the tubular member and the core wire.

23. The method of claim 22, wherein the tubular member comprises a helical coil.

24. The method of claim 22, wherein the tubular member comprises a hypotube.

25. The method of claim 22, wherein the heating includes heating a discrete portion of the tubular member to a temperature at or above the predetermined melting point temperature, and the heated portion of the structural member is allowed to cool on the surface of the elongated shaft such that a discrete connection area is created forming the mechanical bond between the tubular member and the core wire.

26. The method of claim 25, wherein the tubular member includes a perimeter, and the discrete connection area extends about the perimeter of a portion of the tubular member.

27. The method of claim 25, wherein the tubular member includes a perimeter, and the discrete connection extends about only a portion of the perimeter of a portion of the tubular member.

28. The method of claim 22, wherein the core wire comprises a material having a second predetermined melting point temperature above which the material can flow, and the second predetermined melting point temperature is greater than the first predetermined melting point temperature, and wherein the heating includes avoiding heating the core wire to the second predetermined melting point temperature.

29. The method of claim 22, wherein the core wire comprises a metallic material.

30. The method of claim 22, wherein the core wire comprises stainless steel or a nickel-titanium alloy, and wherein the tubular member comprises a nickel-titanium alloy, tungsten, platinum, MP35-N, elgiloy, hastelloy, or combinations or alloys thereof.

31. The method of claim 22, wherein the heating of the portion of the structural member comprises heating with LASER energy.

32. The method of claim 22, wherein the core wire comprises a material, and wherein the mechanical bond between the core wire and the tubular member is achieved without the intermixing in a fluid state of material from the core wire with material from the tubular member.

33. The method of claim 25, wherein a plurality of discrete connection areas are created to form the mechanical bond between the tubular member and the core wire.

34. A method of making a guidewire, the method comprising:
providing an elongated core wire comprising a material and defining an outer surface;
providing a tubular member comprising a material and defining a lumen and having an inner surface;
disposing a portion of the elongated core wire within the lumen of the tubular member such that at least a portion of the inner surface of the tubular member is adjacent the outer surface of the core wire; and
providing means for creating a mechanical bond between the tubular member and the core wire without the intermixing in a fluid state of material from the core wire with material from the tubular member, and without the use of an additional bonding material.

35. A medical device comprising:
an elongated shaft comprising a material and defining a surface;
a structural member comprising a metallic material having a predetermined melting point temperature above which the material can flow, the structural member disposed adjacent the elongated shaft such that at least a portion of the structural member is adjacent the surface; and
a discrete connection area forming a mechanical bond between the structural member and the elongated shaft, the discrete connection area including a portion of the structural member that was heated, allowed to flow onto and allowed to cool on the surface of the elongated shaft without intermixing in a fluid state with material from the elongated shaft.

36. The medical device of claim 35, wherein the structural member comprises a tubular member defining a lumen and having an inner surface, and the structural member is disposed about the elongated shaft such that at least a portion of the elongated shaft is disposed within the lumen of the structural member.

37. The medical device of claim 36, wherein the tubular member comprises a helical coil.

38. The medical device of claim 36, wherein the tubular member comprises a hypotube.

39. The medical device of claim 36, wherein the tubular member includes a perimeter, and the discrete connection area extends about the perimeter of a portion of the tubular member.

40. The medical device of claim 36, wherein the tubular member includes a perimeter, and the discrete connection extends about only a portion of the perimeter of a portion of the tubular member.

41. The medical device of claim 35, wherein the elongated shaft comprises a material having a second predetermined melting point temperature above which the material can flow, and the second predetermined melting point temperature is greater than the first predetermined melting point temperature.

42. The medical device of claim 36, wherein the elongated shaft comprises a material having characteristics that are adversely affected when exposed to a second predetermined temperature, and the second predetermined temperature is greater than the first predetermined melting point temperature.

43. The medical device of claim 35, wherein the elongated shaft comprises a metallic material.

44. The medical device of claim 35, wherein the elongated shaft comprises stainless steel or a nickel-titanium alloy, and wherein the structural member comprises a nickel-titanium alloy, tungsten, platinum, MP35-N, elgiloy, hastelloy, or combinations or alloys thereof.

45. The medical device of claim 35, wherein the discrete connection area was formed by heating of the discrete portion of the structural member with LASER energy.

46. The medical device of claim 35, wherein the discrete connection area is created to form the mechanical bond between the structural member without the use of an additional bonding material.

47. The medical device of claim 35, further including a plurality of discrete connection areas.

48. A guidewire comprising:
an elongated core wire comprising a material and defining an outer surface;
a tubular member defining a lumen and having an inner surface, the tubular member comprising a metallic material having a predetermined melting point temperature above which the material can flow, the tubular member being disposed about a portion of the core wire such that the portion of the core wire extends within the lumen of the tubular member such that at least a portion of the inner surface of the tubular member is adjacent the outer surface of the core wire; and
a discrete connection area forming a mechanical bond between the tubular member and the elongated core wire, the discrete connection area including a portion of the tubular member that was heated, allowed to flow onto and allowed to cool on the surface of the core wire without intermixing in a fluid state with material from the core wire.

49. A guidewire comprising:
an elongated core wire comprising a material and defining an outer surface; and
a tubular member defining a lumen and having an inner surface, the tubular member comprising a metallic material having a predetermined melting point temperature above which the material can flow, the tubular member being disposed about a portion of the core wire such that the portion of the core wire extends within the lumen of the tubular member such that at least a portion of the inner surface of the tubular member is adjacent the outer surface of the core wire;
wherein the tubular member is heat crimped to the elongated core wire such that a mechanical bond if formed between the tubular member and the elongated core wire, wherein the heat crimping is achieved without intermixing in a fluid state of material from the core wire and material from the tubular member.

50. A guidewire comprising:
an elongated core wire comprising a material and defining an outer surface;
a tubular member defining a lumen and having an inner surface, the tubular member comprising a metallic material having a predetermined melting point temperature above which the material can flow, the tubular member being disposed about a portion of the core wire such that the portion of the core wire extends within the lumen of the tubular member such that at least a portion of the inner surface of the tubular member is adjacent the outer surface of the core wire; and
means for connecting the tubular member to the elongated core wire such that a mechanical bond if formed between the tubular member and the elongated core wire, wherein the mechanical bond is achieved without intermixing in a fluid state of material from the core wire and material from the tubular member.
